(19) United States
(12) Patent Application Publication
(10) Pub. No.: US 2021/0116450 A1
(43) Pub. Date: Apr. 22, 2021

(54) RECOMBINANT EXPRESSION OF THE VP2-BETA DOMAIN IN ESCHERICHIA COLI, PURIFICATION AND ITS USE FOR DETECTION OF ANTI-INFECTIOUS BURSAL DISEASE VIRUS ANTIBODIES BY DIPSTICK AND LATERAL FLOW STRIP

(71) Applicants: Moazur Rahman, Faisalabad (PK); Mazhar Iqbal, Faisalabad (PK)

(72) Inventors: Moazur Rahman, Faisalabad (PK); Mazhar Iqbal, Faisalabad (PK)

(21) Appl. No.: 17/064,511

(22) Filed: Oct. 6, 2020

Related U.S. Application Data
(60) Provisional application No. 62/924,091, filed on Oct. 21, 2019.

(51) Int. Cl. G01N 33/558; G01N 33/532; G01N 33/543; G01N 33/68

(52) U.S. Cl. CPC G01N 33/558; G01N 33/6803; G01N 33/54313; G01N 33/532

(57) ABSTRACT

Disclosed herein is the expression of beta-domain (Asp201-Gly339) of the VP2 protein (NCBI Acc. No. KT281984) of infectious bursal disease virus (IBDV), in codon optimized Rosetta 2 (DE3) strain of E. coli followed by its purification by affinity and gel filtration chromatography and use for development of dipstick and lateral flow strip for diagnosis of antibodies produced in chicken upon IBDV infection/vaccination.

### CROSS REFERENCE TO RELATED APPLICATION

This is a Non-Provisional Application of the Provisional U.S. Patent Application Ser. No. 62/924,091 entitled Recombinant Expression Of The VP2-Beta Domain In Escherichia Coli, Purification And Its Use For Detection Of Anti-Infectious Bursal Disease Virus Antibodies By Dipstick And Lateral Flow Strip, filed on Oct. 21, 2019, the contents of which are incorporated herein by reference in its entirety

### FIELD OF THE INVENTION

The present invention relates to diagnosis of infectious bursal disease or anti-IBDV antibodies in poultry birds, broiler and layer chicken.

### BACKGROUND OF THE INVENTION

Poultry is one of the vibrant sectors of agriculture industry in Pakistan. Current investment in this sector is more than 200 B rupees and during 2017-2018, poultry has contributed 1.4% in GDP. Poultry is an organized and fast-growing segment and is contributing 30% of total meat production in the country with 8 to 10% annual increment. It also generates employment for 1.5 million people directly or indirectly. However, this sector faces huge economic losses every year due to infection by several viral diseases including infectious bursal disease (IBD). The disease is endemic in Pakistan and its several periodic outbreaks have been reported in the past and in year 2018 many outbreaks were reported in Punjab, a province of Pakistan. To combat this disease, first step is to develop authentic robust diagnostic assay/test targeting local strains of IBDV. Currently, no diagnostic kit targeting local strains of IBDV is available in Pakistan.

Poultry sector plays a pivotal role in the economy of many countries and meeting the demand of food for the rapidly growing populations especially in developing countries. More than 50 billion chickens are raised annually as a source of meat and eggs worldwide (http://www.ciwf.org.uk/farm-animals/chickens/). Unfortunately, this industry is at the risk of many infectious diseases that cause major losses. Infectious Bursal Disease (IBD) is one of the such common diseases that was first time reported in 1957 in Gumboro, Del., USA and is commonly known as Gumboro disease. Chicken are infected at infancy (2-6 weeks) and become susceptible to bacterial and other viral infections due to weakened immunity. Most of the member states of OIE (Office International des Epizooties) including Pakistan have the prevalence of IBDV.

IBDV can survive in extreme environmental conditions and exists in even highly clean and disinfected areas. It can cause infections in different avian species like chickens, turkeys, ducks and ostriches. However, clinical disease and lesions in the bursa of Fabricus (FB) of chickens are only reported.

There are two known serotypes of IBDV, serotype 1 and serotype 2 that exhibit difference in their virulence to poultry Serotype 1 causes IBD while serotype 2 is non-virulent and has not been found to proliferate in the FB of infected chicken. Both serotypes share same receptors on the cell surface and compete resulting in the loss in detrimental effects of the IBDV serotype 1. There is considerable variation in the pathogenicity of the different strains of IBDV virus. Based on these variations, there are total four types of IBDV, attenuated (with no infectivity but antigenic), virulent, antigenic variant and very virulent strain (vvIBDV). The virus proves to be highly fatal and causes suppression of immunity in poultry which is not protected by any means. Chicken reared for meat (broilers) as well as those reared for eggs (layer chicks) are equally susceptible to the disease.

Efforts continue to control the disease by vaccination considering that IBD is still one of the major diseases. In order to control the disease effectively, a surveillance program must be employed throughout the country that will provide information in which region regular vaccination is needed. However, to initiate such program, sensitive, specific, robust and on-site detection assays targeting local isolate of IBDV should be made available.

The structural and non-structural proteins of pathogens have been exploited as antigen for development of diagnostics assays such as ELISA, immune-chromatographic tests, etc. In the case of IBDV, capsid is comprised of five proteins VP1, VP2, VP3, VP4 and VP5, irrespective of the strain of the virus. Out of these five, VP2 and VP3 are structural proteins. The VP2 is the predominant structural protein with intense antigenic capability and responsible for the induction of programmed cell death in B-lymphocytes and other target cells. Moreover, VP2 is located on the outer surface of the capsid and plays important role in the induction of antibodies which neutralize the virus. Therefore, VP2 can be exploited as an antigen to develop diagnostic assays for the detection of anti-IBDV antibodies.

VP2 is comprised of three domains, namely, protrusion (beta-domain), shell and base domain. The beta-domain (Asp201-Gly339) is consisted of 10 beta sheets and lies on the surface of virus capsid. From the VP2 three dimensional structure, it is evident that beta sheets and loops are independent structural unit and may not require any partner sequence to fold independently in correct conformation as neutralizing antibody induction is very much sensitive to the folding and final conformation of the antigenic site of VP2 protein.

The VP2 being a major antigenic protein of IBDV and its promptness to the variability depending on the geographical distribution, its beta-sheet domain can be exploited as a diagnostic antigen as it may fold independently and located on outer surface of the viral capsid.

Various diagnostic methods are used for virus detection: these include virus isolation from cell culture, agar gel precipitation test, virus neutralization, restriction fragment-length polymorphism, ELISA and reverse transcriptase polymerase chain reaction (RT-PCR). However, these methods have limited application in clinical diagnosis because they are time-consuming and require special equipment and professional skills. Moreover, these cannot be performed in the field and the cost per assay is relatively high.

Currently, various point of care tests (also known as pen-side test or field-based assay) for detection of pathogens are widely used because of their robustness, sensitivity, and specificity and ease of use. Moreover, these do not require any professional expertise and special instrument and can be used equally in clinic and on-site. Dipstick and lateral flow strip devices have been developed for detection of both IBDV-antigen and IBDV antibodies in several countries and also available commercially (http://www.acdiainc.com/Infectious%20Bursal%20Disease%20Virus%20Antigen.html). But none of them make use of recombinant beta-domain of the VP2 protein of IBDV as an antigen. Moreover, the subjected beta-domain allows development of diagnostic devices targeting genetic variability of local IBDV-strains.

Considering the antigenic nature of the surface exposed beta-domain of the VP2 protein, it was produced, in this invention, by recombinant means and purified for development of point-of-care devices (Dipstick and later flow strip) for diagnosis of anti-IBDV antibodies. Dipstick and later flow strip can be used in field and laboratory with same ease of convenience whereas use of most of other diagnostic assays such PCR, ELISA, etc., is restricted to laboratory only.

### BRIEF DESCRIPTION OF THE ACCOMPANYING DRAWINGS

FIG. 1: Structural representation (A) and cloning strategy (B) of expression clone of beta domain of the VP2 protein of IBDV

FIG. 2: Size-exclusion chromatography of the nickel-affinity purified recombinant beta-domain of the VP2 protein. Major peak at around 15 mL position represent major oligomeric form of beta-domain. The inset shows monomer, dimer and the trimeric forms of the beta-domain, analyzed by SDS-PAGE.

FIG. 3: Determination of protein concentration to produce stable beta-domain and gold-nanoparticle conjugates. Beta-domain concentration 0.6 μg/200 μL is taken an optimum concentration to produce stable conjugates.

FIG. 4: Dipstick format: Detection of anti-IBDV antibodies produced in chicken upon infection with IBDV. SPF represents specific pathogen free chicken

FIG. 5: Lateral flow strip: Detection of anti-IBDV antibodies produced in chicken upon infection with IBDV

### DETAILED DESCRIPTION OF THE INVENTION

The present invention relates to the development of a dipstick and a lateral flow strip for the detection of the anti-IBDV antibodies using the recombinant beta-domain of the VP2 protein of IBDV. The VP2 protein is highly immunogenic and capable of capturing anti-IBDV antibodies. Being a major structural and antigenic component of IBDV, the beta-domain of the VP2 protein is represented here as a convincing candidate for the recombinant production and development of diagnostic tools. Another important aspect associated with the present invention is to provide antigen (beta-domain) that was produced from the VP2 sequence of a local (Pakistan) pathogenic very virulent IBDV isolate (Acc. No. KT281984). This use results in high antigen-antibody affinity and specificity in correspondence with diagnosis related to the local vvIBDV strains as compared to the diagnostic tools developed using the whole or the parts of IBDV belong to other geographical regions.

To pursue development of diagnostic assays, several virulent IBDV isolates from a recent outbreak in Punjab were collected and nucleotide sequence of the VP2 gene coding for a viral surface protein was amplified and submitted in GenBank under accession no.—KT28 1 984.

In the proposed project, beta-domain of the VP2 protein was produced in Escherichia coli by recombinant means and purified using combinations of affinity, ion-exchange and/or size exclusion chromatography techniques. Purified protein was used for conjugation with gold-nanoparticles for developing on-site lateral flow immune-strip test or dipstick to detect anti-IBDV antibodies in the infected chicken. It is expected that locally developed diagnostic test targeting the genetic variability of local strains of IBDV will be more effective.

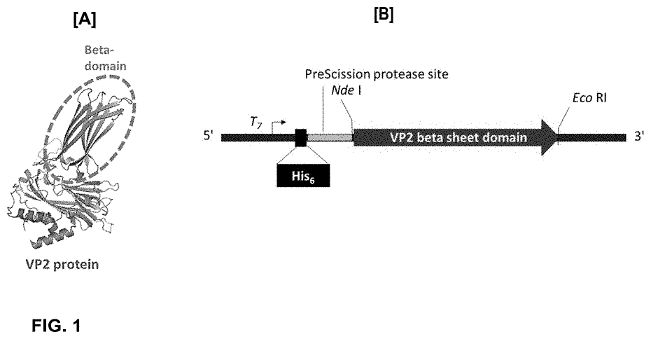

The preferred embodiment of the present invention is to develop tools in the form of dipstick and lateral flow strip using beta-domain of IBDV for diagnosis of anti-IBDV antibodies produced in chicken upon corresponding viral infection or vaccination. To construct dipstick and lateral flow diagnostic tools, in another embodiment, nucleotide sequence corresponding to beta-domain was cloned in an expression vector under the control of T7 promoter (FIG. 1) and expression conditions for synthesis of beta-domain were optimized in a codon optimized Escherichia coli, Rosetta 2 (DE3) using standard procedures.

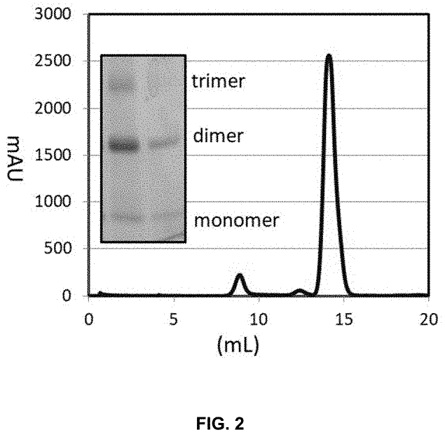

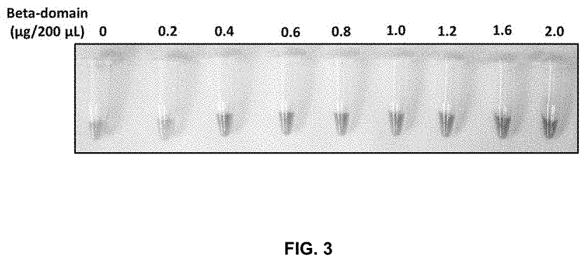

In another embodiment, oligo histidine tagged beta-domain was purified using nickel-affinity and size exclusion chromatographic methods and integrity was verified by sodium dodecyl gel electrophoresis (FIG. 2). Purified protein at an optimized concentration of 0.6 μg/200 μL was used to produce stable beta-domain and gold nanoparticles conjugates (FIG. 3) using NaCl based standard method (https://cdn.shopify.com/s/files/1/0257/8237/files/BioReady_40_nm_Bare_Gold_Passive_Co njugation_Protocol.pdf).

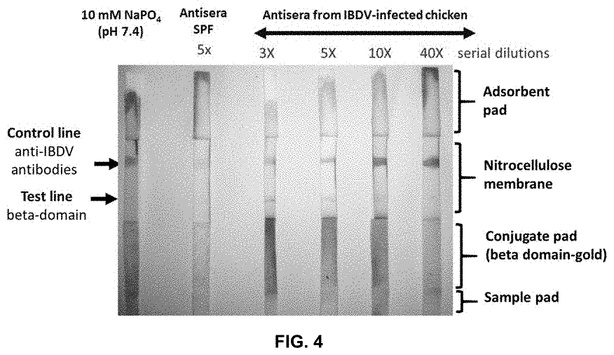

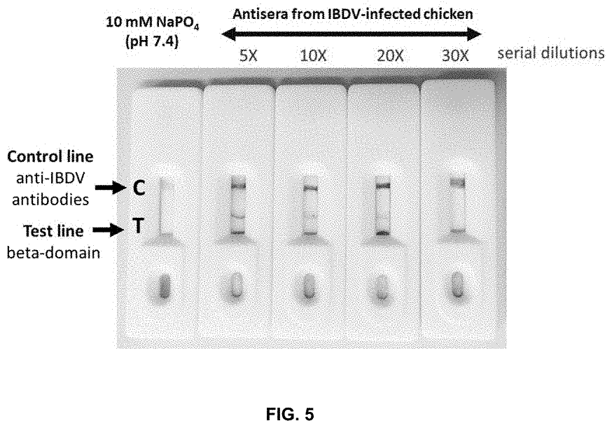

In another preferred embodiments, to construct dipstick and lateral flow strip, technical aspect for precise interaction of antigen (beta-domain conjugated to gold nanoparticles) and anti-IBDV antibodies produced in chicken were optimized. Further, various components conjugate pad treated with beta-domain-gold nanoparticles conjugates, nitrocellulose membrane printed with test (beta-domain) and control (anti-IBDV antibodies), buffer treated adsorbent and sample pad were assembled to fabricate dipstick and lateral flow strip devices as described somewhere. These tools were tested as point-of-care device for detection of anti-IBDV antibodies FIGS. 4 and 5). Briefly, for the detection of IBDV antibodies, serum samples containing anti-IBDV antibodies (collected from field) or specific pathogen free antisera were added drop wise on to the sample pad (or dipstick immersed in diluted serum sample) and then strip/dipstick was incubated laterally to allow the sample to pass through the NC membrane for 5-10 minutes. According to the standard protocol, positive results correspond to appearance of two lines in the test and control region whereas negative result is denoted by the appearance of only one red line in the control region.

What is claimed is:

1. A method of diagnosing presence of antibodies to infectious bursal disease virus (IBDV) by reacting a serum sample with beta-domain (Asp201-Gly339) of a VP2 protein of the IBDV conjugated with gold-nanoparticles as a positive indicator of infection.

2. The method of claim 1, wherein the beta-domain of the VP2 protein is expressed in a codon optimized Rosetta 2 (DE3) strain of E. coli and purified by consecutive affinity, ion-exchange and size exclusion chromatography.

3. The method of claim 1, wherein a test device for diagnosis comprises a dipstick further comprising a buffered sample application pad, a fiberglass pad infused with the beta-domain conjugated with gold-nanoparticles in communication with the application pad, and a nitrocellulose membrane pad marked with a test line of beta-domain non-conjugated protein and a control line of anti-IBDV antibodies, in communication with the fiberglass pad.

4. The method of claim 3, wherein a diagnosis is made by adding a serum sample to the sample pad and incubating the dipstick for 5-10 minutes, placed in a vertical or horizontal position, to enable the serum sample to migrate across the nitrocellulose membrane and concluding a positive test if both test and control lines are visible, and negative if only the control line is visible.
